(19) United States
(12) Patent Application Publication
(10) Pub. No.: US 2013/0225641 A1
(43) Pub. Date: Aug. 29, 2013

(54) ANTICANCER COMPOUNDS AND PREPARATION METHODS THEREOF

(71) Applicants: Banghe Pharmaceutical Co., Ltd., (CN); Nan Zhang, Nanjing (CN); Rong Zhong, Nanjing (CN); Suzhou Maidixian Pharmaceutical Inc.,, (US)

(72) Inventors: Nan Zhang, Nanjing (CN); Rong Zhong, Nanjing (CN)

(21) Appl. No.: 13/698,369

(22) PCT Filed: Aug. 30, 2010
(86) PCT No.: PCT/CN10/76451

§ 371 Date: Apr. 29, 2013

(30) Foreign Application Priority Data
May 18, 2010 (CN) 201010174968.8

(51) Int. Cl. C07D 213/81

(52) U.S. Cl. CPC C07D 213/81
USPC 514/346; 546/291

(57) ABSTRACT

Two new compounds with anticancer effects of N-[4-chloro-3-(trifluoromethyl)phenyl]-[4-(N-methyl-formamide)(4-pyridyloxy)phenyl]-thiourea and N-[4-chloro-3-(trifluoromethyl)phenyl]-[4-(N-methyl-formamide)(4-pyridylthio)phenyl]-thiourea, and salts thereof are disclosed. Preparation methods of the two new compounds and pharmaceutical compositions containing the new compounds are further disclosed. Experimental studies show that the two new compounds can effectively inhibit the activity of Raf and VEGFR protein kinase, widely inhibit growth of various types of human tumor cell lines and further induce apoptosis of tumor cells. Human tumor heterograft model investigation proves that the two new compounds are effective antineoplastic agents, and can sharply inhibit growth of human liver cancer cells, lung cancer cells and intestinal cancer cells in vivo. Furthermore, the anticancer effects of the compounds are much better than that of Sorafenib.

### CROSS-REFERENCE TO RELATED APPLICATIONS

This application is a national stage application under 35 U.S.C. 371 of International Patent Application Serial No. PCT/CN2010/076451, entitled “Anticancer Compounds and Preparation Methods Thereof,” filed Aug. 30, 2010, which claims priority from Chinese Patent Application No. 201010174968.8, filed May 18, 2010, the disclosures of which are hereby incorporated by reference herein in their entirety.

### TECHNICAL FIELD

The present invention relates to anticancer compounds and salts, preparation methods thereof, and pharmaceutical compositions containing the same.

### BACKGROUND

Primary liver cancer is a clinically common malignant tumor characterized by a covert onset, thus most patients have reached the mid-advanced stage when make a definite diagnosis, thereby always losing the opportunity to radical treatment through operation. Only 10-15% of the newly diagnosed patients can have the tumor radically resected, but with a recurrence rate up to 50-80% 5 years after operation. The treatment for advanced patients is quite difficult and has a poor effect. The survival time is short, as indicated by an average survival time of only 3-4 months after diagnosis, and an average 5-year survival rate of only 5%. Consequently, liver cancer is featured by low early diagnosis rate and surgical resection rate, and poor prognosis etc., thus called the “the king of cancers”.

Regarding the therapeutic methods for liver cancer, there are mainly surgical resection, liver transplantation, hepatic artery interventional therapy, percutaneous ablation, systemic chemotherapy, traditional medicine treatment, and so on at present. Scholars abroad and at home persistently endeavour to do the research on the treatment for liver cancer. In China, Academicians Wu Mengchao and Tang Zhaoyou have made great contributions due to their many meaningful explorations carried out in 1970s and 1980s, for example, improvements in operative methods, including diagnosis and treatment of small liver carcinoma, irregular resection and two-stage resection of liver cancer, and so on. For mid-advanced liver cancer, hepatic artery interventional therapy is the most important conservative therapeutic method, and is obviously effective for a part of the patients. However, interventional therapy suffers from many restrictions in clinical applications, and not a few cases do not meet the indications for interventional therapy, for example, interventional therapy cannot be used on those with serious liver and kidney dysfunction, severe ascites, suppressed bone marrow hematopoiesis or coagulation disorders, too large tumors (accounting for over 70% of the liver volume), completely occluded main portal vein tumor thrombus, severe portal hypertension, complicated active peptic ulcer, or distant metastasis. In these cases, although systemic chemotherapy may be employed as a palliative treatment method, liver cancer cells are resistant to a variety of chemotherapeutic drugs, thus traditional chemotherapeutic drugs have a very low therapeutic effect, at the same time, the patients generally experience basic liver diseases such as viral hepatitis and hepatic cirrhosis etc., thus has hepatic dysfunction, and poor tolerance to chemotherapy, and always cannot receive intense chemotherapy. Recently, the use of some new chemotherapeutic drugs such as oxaliplatin, gemcitabine and capecitabine etc. in the treatment of liver cancer is being actively investigated in clinic, and tend to be feasible; however, all of they are still in research which is carrying out, and the final outcome has not been achieved.

Sorafenib is a new type signal transduction inhibitor developed by Bayer AG, Germany, which achieves the purpose of inhibiting the proliferation of tumor cells and anti-angiogenesis by combining two anticancer routes for Raf/MEK/ERK signalling pathway and VEGFR and PDGFR. Sorafenib is a molecular targeted therapeutic drug, which represents an absolutely new therapeutic drug and strategy and method in liver cancer, has a notable effect, and can effectively prolong the survival time of the patients with advanced liver cancer, thus being a breakthrough and milestone in the treatment of advanced liver cancer, and really starting a new era of liver cancer targeted therapy.

We modify the chemical structure of Sorafenib to produce two whole new substances of chemical entities by a chemical molecular modification method, both can widely inhibit the growth of many types of human tumor cell lines, including liver cancer cells, thus the present invention is come out.

### SUMMARY

The purpose of the present invention is to provide two new compounds with anticancer effect and salts thereof, and pharmaceutical compositions containing the same. Another purpose of the present invention is to provide preparation methods of the new compounds.

The present invention provides two new compounds, N-[4-chloro-3-(trifluoromethyl)phenyl]-[4-(N-methyl-formamide)(4-pyridyloxy)phenyl]-thiourea and N-[4-chloro-3-(trifluoromethyl)phenyl]-[4-(N-methyl-formamide)(4-pyridylthio)phenyl]-thiourea, or pharmaceutically acceptable salts thereof.

The structural formulae of the two compounds are as shown by Formulae I and II sequentially:

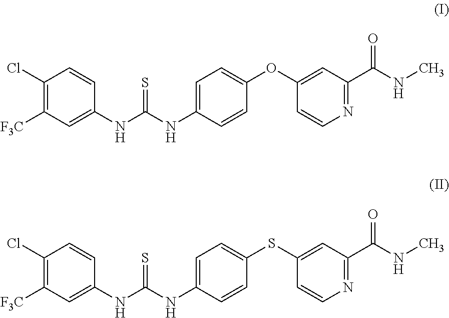 [SMILES: CNC(=O)c1cc(Oc2ccc(NC(=S)Nc3ccc(Cl)c(C(F)(F)F)c3)cc2)ccn1 ; CNC(=O)c1cc(Sc2ccc(NC(=S)Nc3ccc(Cl)c(C(F)(F)F)c3)cc2)ccn1]

For the compound of Formula I, the structure is elucidated through NMR technique and the molecular weight is calculated using MS:

1H-NMR (400 MHz, DMSO) δ 10.159 (s, 1H), 10.182 (s, 1H), 8.777-8.790 (d, J=5.2 Hz, 1H), 8.525-8.539 (d, J=5.6 Hz, 1H), 8.086-8.092 (d, J=2.4 Hz, 1H), 7,804-7.832 (dd, J1=8.8 Hz, J2=2.4 Hz, 1H), 7.671-7.692 (d, J=8.4 Hz, 1H), 7.567-7.589 (d, J=8.8 Hz, 2H), 7.419-7.426 (d, J=2.8 Hz, 1H), 7.221-7.243 (d, J=8.8 Hz, 2H), 7.175-7.195 (dd, J1=5.6 Hz, J2=2.4 Hz, 1H), 2.781-2.793 (d, J=4.8 Hz, 3H). MS 497 [M+H]+.

The reaction scheme for synthesizing the compound of Formula I is as shown below:

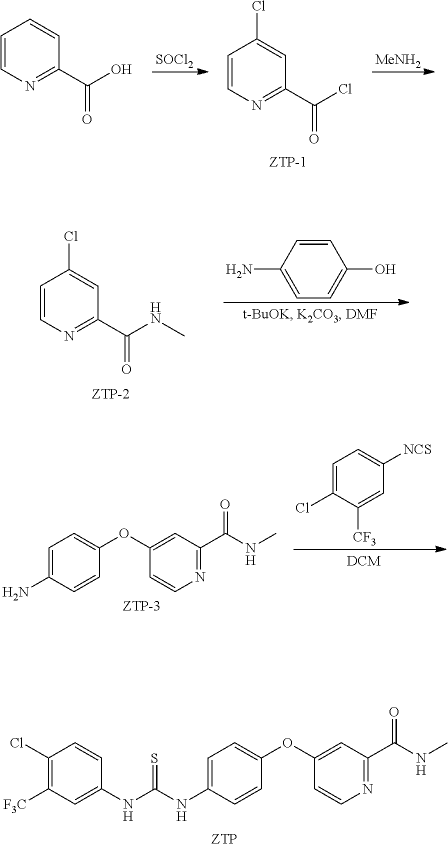

The compound of Formula I is reacted with toluenesulfonic acid, to produce a toluenesulfonate of the compound of Formula I.

For the compound of Formula II, the structure is elucidated through NMR technique and the molecular weight is calculated using MS:

1H-NMR (400 MHz, DMSO) δ 10.300 (s, 1H), 10.370 (s, 1H), 8.738-8.749 (d, J=4.4 Hz, 1H), 8.417-8.430 (d, J=5.2 Hz, 1H), 8.088-8.094 (d, J=2.4 Hz, 1H), 7.807-7.813 (d, J=2.4 Hz, 1H), 7.687-7.726 (m, 3H), 7.591-7.612 (m, 3H), 7.254-7272 (dd, J1=5.2 Hz, J2=2.0 Hz, 1H), 2.762-2.774 (d, J=4.8 Hz, 3H). MS 481 [M+H]+.

The reaction scheme for synthesizing the compound of Formula II is as shown below:

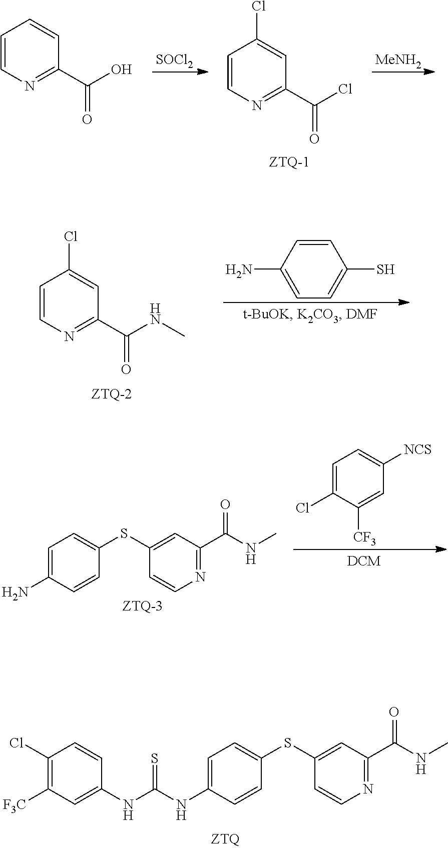

The compound of Formula II is reacted with toluenesulfonic acid, to produce a toluenesulfonate of the compound of Formula II.

The compound of Formula I or II or a salt thereof such as toluenesulfonate, can be formulated with a pharmaceutically acceptable carrier and/or excipient into an anticancer drug, which can be prepared into a tablet, dissolved medicine, capsule, dropping pill, oral solution, or injection. The pharmaceutically acceptable carrier and/or excipient may be selected from cereal oils, carboxymethylcellulose sodium, and so on. For the compound of Formula I or II or a salt thereof, a recommended oral dose is generally 100 mg/m² of body surface area per day, which is orally administered 0.5 hour after breakfast once a day for three consecutive weeks, followed by one-week break, which constitutes one course of treatment. In particular cases, the dose may be adjusted by a physician according to the conditions.

It is found through experimental studies that both N-[4-chloro-3-(trifluoromethyl)phenyl]-[4-(N-methyl-formamide)(4-pyridyloxy)phenyl]-thiourea and N-[4-chloro-3-(trifluoromethyl)phenyl]-[4-(N-methyl-formamide)(4-pyridylthio)phenyl]-thiourea can effectively inhibit the activity of Raf and VEGFR protein kinase, and can widely inhibit the growth of many types of human tumor cell lines, and further induce apoptosis of tumor cells. In human tumor heterograft model investigations, the two new compounds are proved to be effective antitumor agents, and can intensively inhibit growth of human liver cancer cells, lung cancer cells and intestinal cancer cells in vivo. Furthermore, the anticancer effects of the compounds are obviously better than that of Sorafenib.

### BRIEF DESCRIPTION OF DRAWINGS

FIG. 1 is a plot of tumor volume vs. days after transplantation when the compounds ZTP and ZTQ, Sorafenib, and control are used for treating subcutaneously inoculated human liver cancer (HepG3B) tumor cells;

FIG. 2 is a plot of tumor volume vs. days after transplantation when the compounds ZTP and ZTQ, Sorafenib, and control are used for treating subcutaneously inoculated human colon cancer (HCT-116) tumor cells; and

FIG. 3 is a plot of tumor volume vs. days after transplantation when the compounds ZTP and ZTQ, Sorafenib, and control are used for treating subcutaneously inoculated human lung cancer (NCI-H23) tumor cells.

### DETAILED DESCRIPTION

### (1) Preparation of N-[4-chloro-3-(trifluoromethyl)phenyl]-[4-(N-methyl-formamide)(4-pyridyloxy)phenyl]-thiourea

The particular operations were as follows:

### 1. Preparation of 4-chloropyridyl-2-carbonyl chloride (ZTP-1)

Dimethylformamide (2 mL) was added dropwise to a solution of pyridine-2-carboxylic acid (20 g, 0.16 mol) in thionyl chloride (80 mL) at 40° C., stirred at this temperature for 10 min after addition is completed, then heated to 72° C., and stirred overnight. As LC-MS (Liquid Chromatography-Mass Spectrometry) showed that the reaction was still incomplete, additional thionyl chloride (20 mL) was added, and continuously reacted at 72° C. for 3 hrs, LC-MS showed that the reaction was not changed. The reaction solution was cooled to room temperature, thionyl chloride was removed under reduced pressure, toluene (200 mL) was added and the mixture was evaporated to dryness under reduced pressure, and then toluene (30 mL) was added, to give a solution which was directly used in the next reaction;

### 2. Preparation of 4-chloro(2-pyridyl)-N-methylcarboxamide (ZTP-2)

25% aqueous methylamine solution (60 mL) was cooled to −5° C., to which a solution of ZTP-1 (˜60 g) in toluene was added dropwise while the temperature was maintained below 20° C. After addition is completed, the reaction solution was stirred for 1 hr at 20° C. and ethyl acetate (200 mL) and water (50 mL) were added. The organic layer was washed with saturated brine, dried over anhydrous sodium sulfate, and concentrated, to give 20.1 g of an orange oil, which was directly used in the next reaction without purification. The molecular weight of ZTP-2 was calculated using MS. MS 171 [M+H]+.

### 3. Preparation of 4-[(4-aminophenoxy)(2-pyridyl)]-N-methylcarboxamide (ZTP-3)

Under the protection of nitrogen, 4-aminophenol (5.12 g, 46.89 mmol) was dissolved in dimethylformamide (80 ml), potassium tert-butoxide (5.47 g, 48.77 mmol) was added and stirred at room temperature for 2 hrs, and then ZTP-2 (8 g, 46.89 mmol) and potassium carbonate (3.43 g, 24.85 mmol) were added, heated to 80° C., and reacted overnight. TLC (thin-layer chromatography) showed that the reaction was complete. The reaction solution was cooled to room temperature, and water (200 ml) and ethyl acetate (150 ml) were added. The organic layer was sequentially washed with a saturated aqueous sodium carbonate solution and saturated brine, dried over anhydrous sodium sulfate, and concentrated. The residue was subjected to column chromatography (petroleum ether:ethyl acetate=3/1-0/1, V/V) to obtain 4.9 g of a product ZTP-3 as a yellow solid. Yield: 43%. LC-MS showed that there existed a by-product (with chloro at position 6 of pyridine). The molecular weight of ZTP-3 was calculated using MS. MS 244 [M+H]+.

### 4. Preparation of N-[4-chloro-3-(trifluoromethyl)phenyl]-[4-(N-methyl-formamide)(4-pyridyloxy)phenyl]-thiourea (ZTP)

Under the protection of nitrogen, ZTP-3 (2.0 g, 8.2 mmol) and 4-chloro-3-trifluoromethylphenyl isocyanate (2.2 g, 9.8 mmol) were added to dichloromethane (10 ml) at 20° C., and stirred overnight. TLC showed that the reaction was complete. The reaction solution was concentrated, and the residue was purified by column chromatography (dichloromethane:methanol=1/0-200/1-50/1,V/V), to give a yellow crude product. The crude product was dissolved in dichloromethane (3 ml), and then diethyl ether (˜10 ml) was added slowly, to precipitate a crystal out, which was filtered, washed with diethyl ether, and dried, to obtain 2.3 g of ZTP as a white solid. Yield: 59%.

### (2) Preparation of N-[4-chloro-3-(trifluoromethyl)phenyl]-[4-(N-methyl-formamide)(4-pyridyloxy)phenyl]-thiourea toluenesulfonate

To a 1 L reaction flask equipped with a condenser, a stirrer, and a thermometer, ZTP (48 g) and absolute ethanol (750 ml) were added, stirred, and heated to reflux. After being clearly dissolved, toluenesulfonic acid (18 g) was added, and reacted under reflux for about 1 hour and 20 minutes. After the reaction is completed, the reaction solution was cooled to room temperature, filtered, washed with ethanol (30 ml), and dried, to obtain 54 g of a crude product. The crude product was added to distilled water (200 ml), stirred, and heated to reflux. After being clearly dissolved, activated carbon (2 g) was added, and filtered while hot after 10 min. The filtrate was stood at room temperature for about 1 day, filtered, and dried, to obtain 50 g product. Yield: 77%.

### (3) Preparation of N-[4-chloro-3-(trifluoromethyl)phenyl]-[4-(N-methyl-formamide)(4-pyridylthio)phenyl]-thiourea

### 1. Preparation of 4-chloropyridyl-2-carbonyl chloride (ZTQ-1)

Dimethylformamide (2 mL) was added dropwise to a solution of pyridine-2-carboxylic acid (20 g, 0.16 mol) in thionyl chloride (80 mL) at 40° C., stirred at this temperature for 10 min after addition is completed, then heated to 72° C., and stirred overnight. As LC-MS showed that the reaction was still incomplete, additional thionyl chloride (20 mL) was added, and continuously reacted at 72° C. for 3 hrs, LC-MS showed that the reaction was not changed. The reaction solution was cooled to room temperature, thionyl chloride was removed under reduced pressure, toluene (200 mL) was added and the mixture was evaporated to dryness under reduced pressure, and then toluene (30 mL) was added again, to give a solution which was directly used in the next reaction.

### 2. Preparation of 4-chloro(2-pyridyl)-N-methylcarboxamide (ZTQ-2)

25% aqueous methylamine solution (60 mL) was cooled to −5° C., to which a solution of ZTQ-1 (˜60 g) in toluene was added dropwise while the temperature was maintained below 20° C. After addition is completed, the reaction solution was stirred for 1 hr at 20° C. and ethyl acetate (200 mL) and water (50 mL) were added. The organic layer was washed with saturated brine, dried over anhydrous sodium sulfate, and concentrated, to give 20.1 g of an orange oil, which was directly used in the next reaction without purification. The molecular weight of ZTQ-2 was calculated using MS. MS 171 [M+H]+.

### 3. Preparation of 4-[(4-aminophenylthio)(2-pyridyl)]-N-methylcarboxamide (ZTQ-3)

Under the protection of nitrogen, 4-aminothiophenol (5.87 g, 46.89 mmol) was dissolved in dimethylformamide (80 ml), potassium tert-butoxide (5.47g, 48.77 mmol) was added and stirred at room temperature for 2 hrs, and then ZTQ-2 (8 g, 46.89 mmol) and potassium carbonate (3.43 g, 24.85 mmol) were added, heated to 80° C., and reacted overnight. TLC showed that the reaction was complete. The reaction solution was cooled to room temperature, and water (200 ml) and ethyl acetate (150 ml) were added. The organic layer was sequentially washed with a saturated aqueous sodium carbonate solution and saturated brine, dried over anhydrous sodium sulfate, and concentrated. The residue was subjected to column chromatography (petroleum ether:ethyl acetate=3/1-0/1, V/V) to obtain 6.5 g of a product ZTQ-3 as a yellow solid. Yield: 54.2%. LC-MS showed that there existed a by-product (with chloro at position 6 of pyridine). The molecular weight of ZTQ-3 was calculated using MS. MS 260 [M+H]+.

### 4. Preparation of N-[4-chloro-3-(trifluoromethyl)phenyl]-[4-(N-methyl-formamide)(4-pyridylthio)phenyl]-thiourea (ZTQ)

Under the protection of nitrogen, ZTQ-3 (2.0 g, 7.7 mmol) and 4-chloro-3-trifluoromethyl phenyl isocyanate (2.0 g, 9.3 mmol) were added to dichloromethane (10 ml) at 20° C., and stirred overnight. As TLC showed that the reaction was still incomplete, additional 4-chloro-3-trifluoromethylphenyl isocyanate (0.5 g) was added, and continuously reacted for 5 hrs, TLC showed that the raw materials was completely consumed. The reaction solution was concentrated, and the residue was purified by column chromatography (dichloromethane:methanol=1/0-200/1-50/1, V/V), to obtain a yellow crude product. The crude product was dissolved in dichloromethane (3 ml), and then diethyl ether (˜10 ml) was slowly added, to precipitate a crystal out, which was filtered, washed with diethyl ether, and dried, to obtain 1.9 g of ZTQ as a white solid. Yield: 50%.

### (4) Preparation of N-[4-chloro-3-(trifluoromethyl)phenyl]-[4-(N-methyl-formamide)(4-pyridylthio)phenyl]-thiourea (ZTQ) toluenesulfonate

To a 1 L reaction flask equipped with a condenser, a stirrer, and a thermometer, ZTP (50 g) and absolute ethanol (750 ml) were added, stirred, and heated to reflux. After being clearly dissolved, toluenesulfonic acid (18 g) was added, and reacted under reflux for about 1 hour and 20 minutes. After reaction is completed, the reaction solution was cooled to room temperature, filtered, washed with ethanol (30 ml), and dried, to obtain 57 g of a crude product. The crude product was added to distilled water (200 ml), stirred, and heated to reflux. After being clearly dissolved, activated carbon (2 g) was added, and filtered while hot after 10 min. The filtrate was stood at room temperature for about 1 day, filtered, and dried, to obtain 52 g product. Yield: 77.6%.

(1)-(4) above are preparation examples, and (5)-(9) below are test examples.

### (5) Inhibition of Activity of Raf and VEGFR Protein Kinases by Compounds ZTP and ZTQ

Raf is a proto-oncogenic product, and activation of Raf is regulated with stimulation from extracellular cell growth factors. Once activated, Raf can activate MEK through phosphorylation, and further transmits an activation signal to ERK, thereby promoting the growth of tumor cells. In this test, the level of phosphorylated MEK in cells is indicative of the activity of Raf protein kinase in cells. MDA-MB-321 is a human derived breast cancer cell line, which is rich in Raf protein. We used MDA-MB-321 cells to detect whether ZTP and ZTQ can inhibit the activity of Raf protein kinase. MDA-MB-321 cells were subjected to adherent growth in the RPMI 1640 medium (GIBCO-BRL) containing 10% bovine calf serum at 37° C. in 5% CO₂. The cells were treated with various concentrations of ZTP and ZTQ (0, 10 ng/ml, 100 ng/ml, 500 ng/ml, 1 μg/ml, and 10 μg/ml), and the cell proteins were collected 1 hour after treatment. The protein content was determined through Bradford method, and then the level of phosphorylated MEK was assayed through western blotting. Immunoblotting experiment: denatured polyacrylamide gel was used for separation of the proteins with a sample load of 60 μg of total proteins. The proteins were electro-transferred onto a PVDF membrane and blocked. Then mouse anti-human anti-phospho-MEK antibody (1:1000 dilution) (purchased from Cell Signalling Technology, Boston, USA), and horseradish peroxidase labelled goat anti-mouse IG antibody (1:5000 dilution) (purchased from Cell Signalling Technology, Boston, USA) were added, and the response was enhanced by a chemiluminescence reagent. The membrane was exposed to an X-ray film, and the results were analyzed by a gel imaging system. After washing, detection with non-phosphorylated MEK antibody was conducted to correct the result.

The results show that the median inhibitory concentrations (IC₅₀) of ZTP and ZTQ for inhibiting the activity of Raf protein kinase are respectively 45 nM and 20 nM.

VEGFR is a vascular endothelial growth factor receptor regulating the growth, migration, and differentiation of vascular endothelial cells, and determining the start of angiogenesis of tumors. After being stimulated by vascular endothelial cell growth factors, VEGFR forms a dimer, activates its kinase, and makes themselves phosphorylated. The level of the activity of VEGFR protein kinase is detected according to the phosphorylated degree of VEGFR. In this test, we used human microvascular endothelial cells (HUMECs) to determine whether ZTP and ZTQ can inhibit the activity of VEGFR protein kinase. HUMEC cells were subjected to adherent growth in DMEM medium (GIBCO-BRL) containing 10% bovine calf serum at 37° C. in 5% CO₂. The cells were treated with various concentrations of ZTP and ZTQ (0, 10 ng/ml, 100 ng/ml, 500 ng/ml, 1 μg/ml, and 10 μg/ml), and the cell proteins were collected 1 hour after treatment. The protein content was determined through Bradford method, and then the level of phosphorylated VEGFR was assayed through western blotting. Immunoblotting experiment: denatured polyacrylamide gel was used for separation of the proteins with a sample load of 60 μg of total proteins. The proteins were electro-transferred onto a PVDF membrane and blocked. Then mouse anti-human anti-phospho-VEGFR antibody (1:1000 dilution) (purchased from Cell Signalling Technology, Boston, USA), and horseradish peroxidase labelled goat anti-mouse IG antibody (1:5000 dilution) (purchased from Cell Signalling Technology, Boston, USA) were added, and the response was enhanced by a chemiluminescence reagent. The membrane was exposed to an X-ray film, and the results were analyzed by a gel imaging system. After washing, detection with non-phosphorylated VEGFR antibody was conducted to correct the result.

The results show that the median inhibitory concentrations (IC₅₀) of ZTP and ZTQ for inhibiting the activity of VEGFR protein kinase are respectively 21 nM and 9 nM.

### (6) Compounds ZTP and ZTQ Widely Inhibit Human Tumor Cell Lines, but Have No Effect on Normal Cell Lines

The cytotoxicity of ZTP and ZTQ was analyzed by using human tumor cell lines. Human tumor cell lines were purchased from ATCC and NCI, and cultured in DMEM medium containing 10% FBS at 37° C. in 5% CO₂ in an incubator. The cells grown to confluence were used for cytotoxicity analysis. The cells were digested with trypsin, washed with the culture medium, and then counted. 33 tumor cell lines were cultured. The cells were seeded at a density of 3000-6000 cells/well in a 96-well plate and incubated for 16-24 hrs. Then, various concentrations of ZTP or ZTQ dissolved in DMSO were added, and cultured for 72 hrs. The drug-treated cells and control cells were analyzed by MTT.

The results are as shown in Table 1. Table 1 shows that most the human tumor cells are susceptible to the test compounds ZTP and ZTQ. The EC50 of the test compounds for some tumor cells is lower than 1 μM. The cells that are most susceptible to the test compounds ZTP and ZTQ are HUMECs, and the test compounds ZTP and ZTQ are extremely potent tumor angiogenesis inhibitors for HUMECs. In addition, normal human mammary epithelial cells (MCF-10a) and normal mouse fibroblasts (MEFs) are insusceptible to the test compounds ZTP and ZTQ, and these cells still grow well even at a concentration up to 30 μm of the two compounds.

TABLE 1
In-vitro inhibition of growth of human tumor cells by compounds
ZTP and ZTQ

| | | LC50 | |
|---|---|---|---|
| Human tumor cell | | of compound | LC50 of compound |
| lines_{0}} | Cell types | ZTP, (μM) | ZTQ, (μM) |
| LnCAP | Prostatic | 1.05 | 0.82 |
| D145 | Prostatic | 1.59 | 1.23 |
| PC3 | Prostatic | 1.31 | 1.05 |
| HCT-116 | Colonic | 0.87 | 0.76 |
| Widr | Colonic | 0.95 | 0.81 |
| HT29 | Colonic | 1.19 | 0.92 |
| LoVo | Colonic | 1.31 | 1.1 |
| CCL-225 | Colonic | 1.98 | 1.43 |
| CCL-247 | Colonic | 2.41 | 1.7 |
| NCI-H23 | Lung | 0.89 | 0.71 |
| A549 | Lung | 1.22 | 1.01 |
| MDA-MB-231 | Breast | 1.19 | 1.03 |
| MDA-MB-435 | Breast | 1.37 | 1.12 |
| AU-565 | Breast | 1.89 | 1.37 |
| BT-549 | Breast | 1.09 | 0.89 |
| MCF-7 | Breast | 2.21 | 1.7 |
| Caki-1 | Renal | 1.12 | 0.91 |
| ACHN | Renal | 1.61 | 1.21 |
| 786-O | Renal | 1.75 | 1.26 |
| SN12C | Renal | 3.21 | 2.2 |
| SKOV3 | Ovarian | 1.19 | 0.98 |
| IGROV1 | Ovarian | 0.92 | 0.73 |
| Mid PaCa-2 | Pancreatic | 1.25 | 0.91 |
| U-251 | Glioblastoma | 3.67 | 2.5 |
| SK-MEL-5 | Skin (melanoma) | 1.99 | 1.54 |
| G-361 | Skin (melanoma) | 1.91 | 1.36 |
| MCF-10a | Normal mammary | >30 | >30 |
| | epithelial cell | | |
| SGC-7901 | Gastric | 1.31 | 1.03 |
| EC109 | Esophageal | 1.48 | 1.12 |
| CNE-2Z | Nasopharyngeal | 1.89 | 1.31 |
| HepG3B | Liver | 1.71 | 1.23 |
| HUMEC | Human | 0.031 | 0.019 |
| | microvascular | | |
| | endothelial cell | | |
| MEF | Normal mouse | >30 | >30 |
| | fibroblast | | |

### (7) Pharmacokinetic Study of Compounds ZTP and ZTQ in Mice

BALB/c mice (2 males and 2 females) of 8 weeks old were administered with the compounds ZTP and ZTQ (formulated with 15% Captsol or a carrier) at a single dose of 100 mg/kg. 0.5, 1, 3, 6, 12, 24, 48, and 72 hrs after administration, blood was taken from ophthalmic vein and plasma was prepared therefrom, to determine the plasma concentrations of ZTP and ZTQ. The pharmacokinetic parameters of the compounds ZTP and ZTQ given orally by gavage in the 4 mice are summarized as below (Tables 2 and 3):

TABLE 2
Pharmacokinetic parameters of the compound ZTP in mice

| Pharmacokinetic parameters | Mean +/− SD |
|---|---|
| T½ (hour) | 4.1 +/− 0.26 |
| Tmax (min) | 6.4 +/− 7.5 |
| Cmax (uM) | 51 +/− 5.7 |

TABLE 3
Pharmacokinetic parameters of the compound ZTQ in mice

| Pharmacokinetic parameters | Mean +/− SD |
|---|---|
| T½ (hour) | 4.2 +/− 0.24 |
| Tmax (min) | 6.6 +/− 7.6 |
| Cmax (uM) | 52 +/− 5.5 |

### (8) Acute Toxicity Studies of the Compounds ZTP and ZTQ in Mice After Administration

Two groups of mice of 8 weeks old (10/group, 5 males and 5 females) were respectively administered with ZTP and ZTQ (formulated with 15% Captsol or a carrier) at a single dose of 500 mg/kg and divided doses of 100 mg/kg, then respectively observed for 1 and 4 weeks, and weighed every two days. After tests, the mice were sacrificed for pathological analysis.

The results show that no obvious toxicity was observed for the compounds ZTP and ZTQ administered at a single dose of 500 mg/kg and divided doses of 100 mg/kg. All the test mice grow well and no death occurs. Therefore, the compounds ZTP and ZTQ are new broad-spectrum antitumor drugs having broad development prospects. The compounds ZTP and ZTQ are useful as crude drugs for antitumor drugs, and preferably for anti-malignant solid tumor drugs. The compounds ZTP and ZTQ may also be formulated with a pharmaceutically acceptable carrier and/or excipient into antitumor drugs in different dosage forms, including tablets, dissolved medicines, capsules, dropping pills, oral solutions, or injections. The pharmaceutically acceptable carrier and/or excipient include, for example, cereal oils and carboxymethylcellulose sodium etc.

A recommended oral dose for the compounds ZTP and ZTQ is generally 100 mg/m² of body surface area per day, which is administered for three consecutive weeks, followed by one-week break, thereby constituting one course of treatment. The total daily dosage of the compounds ZTP and ZTQ is orally administered 0.5 hour after breakfast once a day, in particular cases, the dose may be adjusted by a physician according to the conditions.

### (9) Effectiveness Study of the Compounds ZTP and ZTQ in a Transplantation Model of Human Tumor in Nude Mice

T-cell deficient nude mice (nu/nu) of 6 weeks old were purchased from Charles River Laboratory, and housed and treated in a sterile environment following Institutional Animal Care & Use Committee Program. 48 mice were respectively subcutaneously inoculated on ribbed belly with 5×10⁶ human liver cancer cells (HepG3B), human lung caner cells (NCI-H23), and human colon cancer cells (HCT-116) suspended in 0.2 ml HBSS/matrigel (50:50, V/V). When the tumor grew to have an average diameter of 7-8 mm and a volume of about 100-200 mm³, 24 mice were picked up, and assigned to treatment groups (16 animals/2 groups), and a control group (8 animals). The 2 treatment groups were administered with the compounds ZTP and ZTQ dissolved in 15% Captsol respectively at a dose of 30 mg/kg and 60 mg/kg, and the control group only received an excipient (15% Captsol).

To ensure that the tumor size had a substantially identical distribution in the treatment groups with the compounds ZTP and ZTQ and the control group at the beginning of the treatment, the mice were classified into three types: small-volume tumors (<4 mm), moderate-volume tumors (4-8 mm), and large-volume tumors (>8 mm). The same number of mice of each type was assigned to the control group and the treatment groups with the compounds ZTP and ZTQ respectively.

The dose schedule of the test compounds in the tumor transplanted nude mice was as follows: the compounds ZTP and ZTQ dissolved in 15% Captsol were daily orally administered as a 200 μl solution containing 0.75 mg of ZTP and 1.5 mg of ZTQ for 5 days/week over 3 consecutive weeks, followed by one-week break; and the control group was only administered with 200 μl of the placebo (15% Captsol) for 3 weeks, followed by 1 week of break. The two groups of animals were housed separately, and allowed to free access to food.

Evaluation of antitumor effect: the tumor size was measured every 3 or 4 days. The tumor volume was calculated using the equation below: V=(a×b)/2, in which a denotes the width (the shorter diameter), and b denotes the length (the longer diameter). Relative tumor volume (RTV) of each tumor refers to the ratio of the volume at a specified time to that at the beginning of the treatment. The average RTV for each treatment group was calculated. The antitumor activity was determined by calculating the tumor growth inhibition using a formula below.

TGI (%)=T/C×100

Where T denotes the average RTV of the treatment group at the end of the experiment, and C denotes the average RTV of the control group. The standard for minimum level of antitumor activity (T/C 42%) formulated by National Cancer Institute was employed. The tumor was resected after experiment, and fixed in methanol.

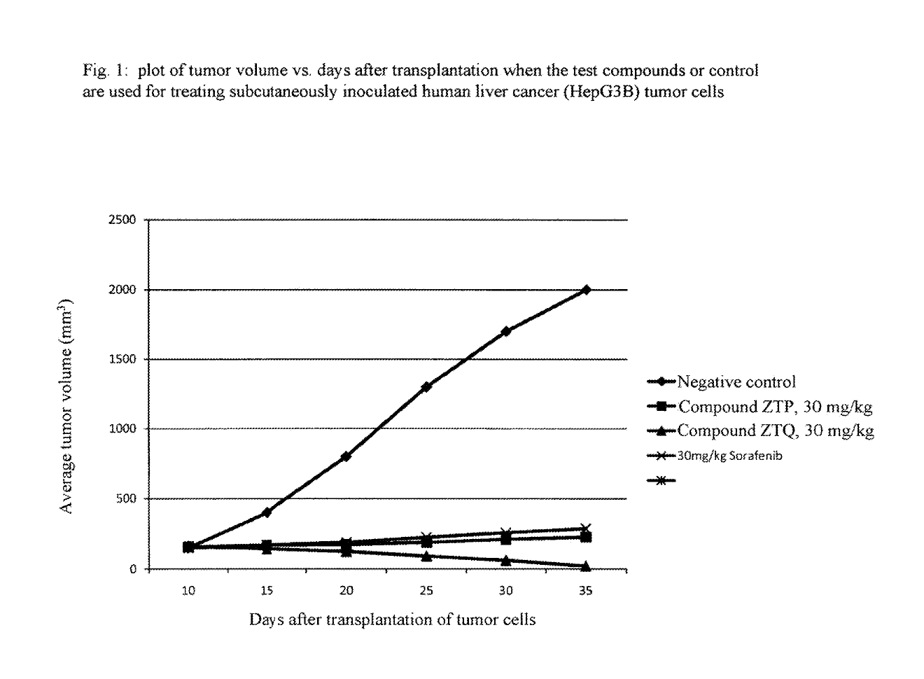

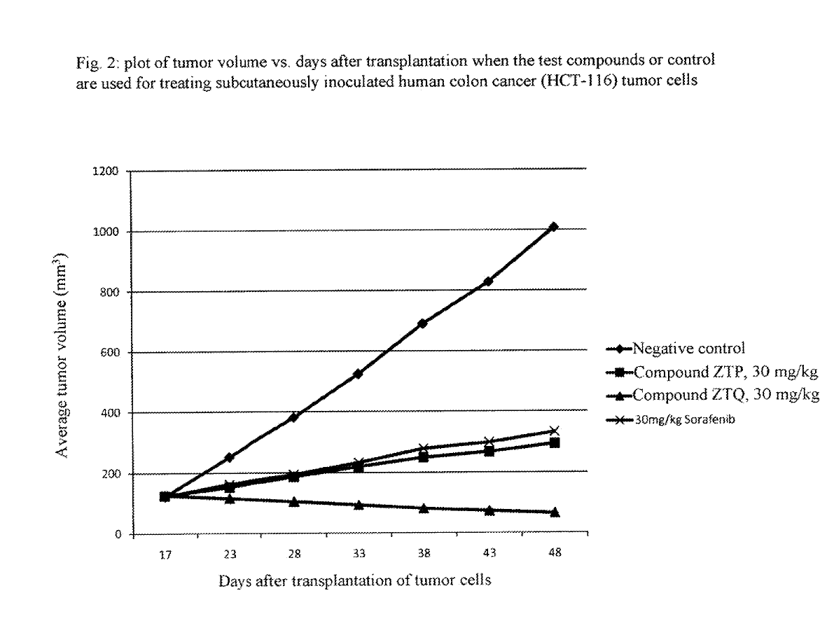

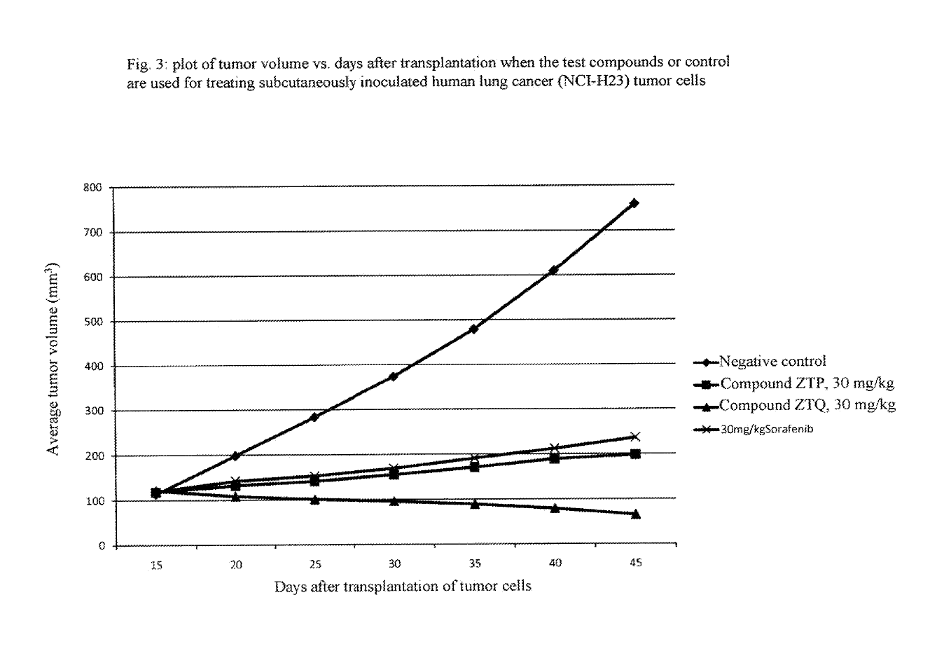

Based on the fact that the compounds ZTP and ZTQ could effectively inhibit the in-vitro proliferation of a variety of tumor cells and human microvascular endothelial cell, the antitumor effect of the compounds ZTP and ZTQ was evaluated using human tumor transplanted nude mice (subcutaneously inoculated), in which the test tumor cells included human liver cancer cells (HepG3B), human colon cancer cells (HCT-116) and human lung cancer cells (NCI-H23). The results show that the TGI values of ZTP, ZTQ, and Sorafenib for human liver cancer cells (HepG3B) after administration were respectively 13%, 1.75%, and 14%; the TGI values of ZTP, ZTQ, and Sorafenib for human colon cancer cells (HCT-116) after administration were respectively 29.2%, 6.45%, and 33%; and the TGI values of ZTP, ZTQ, and Sorafenib for human lung cancer cells (NCI-H23) after administration were respectively 10%, 9.21%, and 11.25%. At the same time, no obvious side effect of ZIT and ZTQ was found, including the change in body weight. In contrast, Sorafenib has a relatively large side effect on the test mice. FIGS. 1-3 show plots of tumor volumes vs. days after transplantation when the compounds ZTP and ZTQ, Sorafenib, and control are used for treating human liver cancer cells (HepG3B), human colon cancer cells (HCT-116) and human lung cancer cells (NCI-H23). The data strongly suggests that the compounds ZTP and ZTQ can intensively inhibit the growth of the three tumors, and ZTP and ZTQ will be new antitumor drugs superior to Sorafenib. Because Sorafenib can be reacted with toluenesulfonic acid to produce Sorafenib toluenesulfonate, and the antitumor effect of Sorafenib toluenesulfonate is obviously stronger than that of Sorafenib. Accordingly, we speculated that the salts formed by reacting ZIT and ZTQ with toluenesulfonic acid will also have an antitumor effect stronger than that of ZTP and ZTQ themselves, and thus the salts of ZTP and ZTQ will also be new effective antitumor drugs.

1. A compound represented by Formula I, or a pharmaceutically acceptable salt thereof: 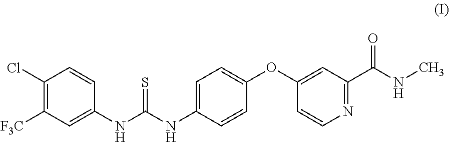 [SMILES: CNC(=O)c1cc(Oc2ccc(NC(=S)Nc3ccc(Cl)c(C(F)(F)F)c3)cc2)ccn1]
wherein the name of the compound of Formula I is N-[4-chloro-3-(trifluoromethyl)phenyl]-[4-(N-methyl-formamide)(4-pyridyloxy)phenyl]-thiourea.

2. A method for preparing the compound of claim 1, the method comprising:
(1) adding dimethyl formamide dropwise to a solution of pyridine-2-carboxylic acid in thionyl chloride at 40° C., stirring, then heating to 72° C., and stirring overnight; and cooling to room temperature after the reaction is completed, removing thionyl chloride under reduced pressure, adding toluene, evaporating to dryness under reduced pressure, and then adding toluene again, to obtain a solution of 4-chloropyridyl-2-carbonyl chloride in toluene;
(2) adding the solution of 4-chloropyridyl-2-carbonyl chloride in toluene dropwise to an aqueous methylamine solution cooled to −5° C., and stirring when the temperature is below 20° C.; and then adding ethyl acetate and water, washing the organic layer with saturated saline, drying it over anhydrous sodium sulfate, and then concentrating to an orange oil, to obtain 4-chloro(2-pyridyl)-N-methylcarboxamide;
(3) Under the protection of nitrogen, dissolving 4-aminophenol in dimethylformamide, then adding potassium tert-butoxide, and stirring at room temperature; then adding 4-chloro(2-pyridyl)-N-methylcarboxamide and potassium carbonate, heating to 80° C., and reacting overnight; and cooling to room temperature after the reaction is completed, adding water and ethyl acetate, washing the organic layer sequentially with a saturated aqueous sodium carbonate solution and saturated saline, drying it over anhydrous sodium sulfate, concentrating, and subjecting the residue to column chromatography (petroleum ether:ethyl acetate=3/1^{˜}0/1, V/V), to obtain a product 4-[(4-aminophenoxy)(2-pyridyl)]-N-methylcarboxamide as a yellow solid; and
(4) Under the protection of nitrogen, adding 4-[(4-aminophenoxy)(2-pyridyl)]-N-methylcarboxamide and 4-chloro-3-trifluoromethylphenyl isocyanate into dichloromethane at 20° C. and stirring overnight; concentrating the reaction solution after the reaction is completed, and subjecting the residue to column chromatography (dichloromethane:methanol=1/0^{˜}200/1^{˜}50/1, V/V), to obtain a yellow crude product; and dissolving the yellow crude product in dichloromethane and adding diethyl ether, to precipitate a crystal out, which is filtered, washed with diethyl ether and dried, to obtain said compound of Formula I, N-[4-chloro-3-(trifluoromethyl)phenyl]-[4-(N-methyl-formamide)(4-pyridyloxy)phenyl]-thiourea.

3. A method for preparing a toluenesulfonate of the compound of claim 1, the method comprising:
adding absolute ethanol and the compound of Formula I N-[4-chloro-3-(trifluoromethyl)phenyl]-[4-(N-methyl-formamide)(4-pyridyloxy)phenyl]-thiourea into a reaction flask, stirring, and heating to reflux; after being clearly dissolved, adding toluenesulfonic acid, and reacting under reflux; cooling to room temperature after the reaction is completed, filtering, washing with ethanol, and drying to obtain a crude product; adding the crude product to distilled water, stirring, and heated to reflux; after being clearly dissolved, adding activated carbon, and filtering while hot; and standing the filtrate at room temperature, filtering, and drying, to obtain a toluenesulfonate of the compound of Formula I.

4. A compound represented by Formula II, or a pharmaceutically acceptable salt thereof: 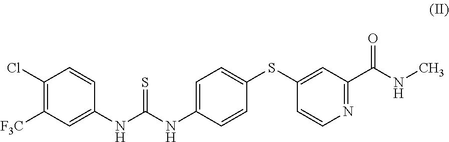 [SMILES: CNC(=O)c1cc(Sc2ccc(NC(=S)Nc3ccc(Cl)c(C(F)(F)F)c3)cc2)ccn1]
wherein the name of the compound of Formula II is N-[4-chloro-3-(trifluoromethyl)phenyl]-[4-(N-methyl-formamide)(4-pyridylthio)phenyl]-thiourea.

5. A method for preparing the compound of claim 4, the method comprising:
(1) adding dimethyl formamide dropwise to a solution of pyridine-2-carboxylic acid in thionyl chloride at 40° C., stirring, then heating to 72° C., and stirring overnight; and cooling to room temperature after the reaction is completed, removing thionyl chloride under reduced pressure, adding toluene, evaporating to dryness under reduced pressure, and then adding toluene again, to obtain a solution of 4-chloropyridyl-2-carbonyl chloride in toluene;
(2) adding the solution of 4-chloropyridyl-2-carbonyl chloride in toluene dropwise to an aqueous methylamine solution cooled to −5° C. and stirring when the temperature is below 20° C.; and then adding ethyl acetate and water, washing the organic layer with saturated saline, drying it over anhydrous sodium sulfate, and then concentrating to an orange oil, to obtain 4-chloro(2-pyridyl)-N-methylcarboxamide;
(3) Under the protection of nitrogen, dissolving 4-aminothiophenol in dimethylformamide, then adding potassium tert-butoxide, stirring at room temperature, then adding said 4-chloro(2-pyridyl)-N-methylcarboxamide and potassium carbonate, heating to 80° C., and reacting overnight; and cooling to room temperature after the reaction is completed, adding water and ethyl acetate, washing the organic layer sequentially with a saturated aqueous sodium carbonate solution and saturated saline, drying it over anhydrous sodium sulfate, concentrating, and subjecting the residue to column chromatography (petroleum ether:ethyl acetate=3/1^{˜}0/1, V/V), to obtain a product 4-[(4-aminophenylthio)(2-pyridyl)]-N-methylcarboxamide as a yellow solid; and
(4) Under the protection of nitrogen, adding 4-[(4-aminophenylthio)(2-pyridyl)]-N-methylcarboxamide and 4-chloro-3-trifluoromethylphenyl isocyanate to dichloromethane at 20° C. and stirring overnight; concentrating the reaction solution after the reaction is completed, and subjecting the residue to column chromatography (dichloromethane:methanol=1/0^{˜}200/1^{˜}50/1, V/V) to obtain a yellow crude product; and dissolving the yellow crude product in dichloromethane and adding diethyl ether, to precipitate a crystal out, which is filtered, washed with diethyl ether and dried, to obtain the compound of Formula II, N-[4-chloro-3-(trifluoromethyl)phenyl]-[4-(N-methyl-formamide)(4-pyridylthio)phenyl]-thiourea.

6. A method for preparing a toluenesulfonate of the compound of claim 4, the method comprising: adding absolute ethanol and the compound of Formula II N-[4-chloro-3-(trifluoromethyl)phenyl]-[4-(N-methyl-formamide)(4-pyridylthio)phenyl]-thiourea to a reaction flask, stirring, and heating to reflux; after being clearly dissolved, adding toluenesulfonic acid, and reacting under reflux; cooling to room temperature after the reaction is completed, filtering, washing with ethanol, and drying to obtain a crude product; adding the crude product to distilled water, stirring, and heated to reflux; after being clearly dissolved, adding activated carbon, and filtering while hot; and standing the filtrate at room temperature, filtering, and drying, to obtain a toluenesulfonate of the compound of Formula II.

7. Use of the compound of claim 1 or a pharmaceutically acceptable salt thereof in the preparation of anticancer drugs.

8. A pharmaceutical composition, comprising the compound of claim 1 or a pharmaceutically acceptable salt thereof as an effective ingredient, and a conventional pharmaceutically acceptable carrier and/or excipient.

9. The pharmaceutical composition according to claim 8, wherein the pharmaceutical composition is prepared into tablets, dissolved medicines, capsules, dropping pills, oral solutions, or injections.

10. The pharmaceutical composition according to claim 8, wherein the pharmaceutical carrier and/or excipient comprises cereal oils, and carboxymethyl cellulose sodium.

11. Use of the compound of claim 4 or a pharmaceutically acceptable salt thereof in the preparation of anticancer drugs.

12. A pharmaceutical composition, comprising the compound of claim 4 or a pharmaceutically acceptable salt thereof as an effective ingredient, and a conventional pharmaceutically acceptable carrier and/or excipient.

13. The pharmaceutical composition according to claim 12, wherein the pharmaceutical composition is prepared into tablets, dissolved medicines, capsules, dropping pills, oral solutions, or injections.

14. The pharmaceutical composition according to claim 8, wherein the pharmaceutical carrier and/or excipient comprises cereal oils, and carboxymethyl cellulose sodium.
